(19) United States
(12) Patent Application Publication
(10) Pub. No.: US 2019/0183566 A1
(43) Pub. Date: Jun. 20, 2019

(54) INTERVENTIONAL ABLATION DEVICE WITH TISSUE DISCRIMINATING CAPABILITY

(71) Applicant: KONINKLIJKE PHILIPS N.V., Eindhoven (NL)

(72) Inventors: Bernardus Hendrikus Wilhelmus Hendriks, Eindhoven (NL); Gerhardus Wilhelmus Lucassen, Eindhoven (NL); Rami Nachabe, Cincinatti, OH (US); Waltherus Cornelis Jozef Bierhoff, Veldhoven (NL); Adrien Emmanuel Desjardins, Waterloo, CA (US)

(21) Appl. No.: 16/282,349

(22) Filed: Feb. 22, 2019

Related U.S. Application Data
(63) Continuation of application No. 13/578,883, filed on Aug. 14, 2012, now abandoned.

(30) Foreign Application Priority Data
Feb. 26, 2010 (EP) 10154770.1

(51) Int. Cl. A61B 18/14; A61B 5/00

(52) U.S. Cl. CPC A61B 18/1477; A61B 2034/2063; A61B 5/0084

(57) ABSTRACT

An interventional ablation device (1) is proposed to comprise an ablation needle (3) with an elongated body (5) and a handle (7). Adjacent to an ablation element (9) provided on the body (5), at least one or preferably two or more sensors (11-21) are provided, preferably at both of opposing sides of the ablation element (9). The ablation device (1) is adapted for detecting physiological information of tissue (27, 29, 31) surrounding an ablation site (33) based on measurement values provided by the sensors. E.g., optical sensors may be used to measure a reflectance spectrum indicating whether the adjacent tissue is healthy tissue (31), tumorous tissue (27) or ablated tissue (29). Using such information, an ablation process may be controlled.

### CROSS-REFERENCE TO PRIOR APPLICATIONS

This application is a continuation of prior application Ser. No. 13/578,883, filed Aug. 14, 2012. Ser. No. 13/578,883 is a U.S. National Phase application under 35 U.S.C. § 371 of International Application Serial No. 2011IB50727, filed on Feb. 22, 2011, which claims the benefit of European Patent Application No. 20100154770, filed on Feb. 26, 2010. These applications are hereby incorporated by reference herein.

### FIELD OF THE INVENTION

The present invention relates to an interventional ablation device which may be used for example for ablating tumorous tissue within a body of a patient. Furthermore, the present invention relates to an interventional ablation needle, to a computer program element enabling to control an ablation procedure and a computer readable medium with such computer program element.

### BACKGROUND OF THE INVENTION

In oncology ablation of tumors is a common procedure especially in cases where resection of the tumor is difficult or almost impossible. For example, in the liver when there are plural tumor sites, complete removal may not be possible when these sites are present in different parts of the liver. What is typically done then is that part of the liver containing the major tumor sites is removed while the remaining part of the liver, also containing tumor sites, is treated by a needle ablation procedure. Therein, one or more needles may be positioned within the tumorous tissue for example by using image guidance based on for example previously or simultaneously acquired ultrasound or computer tomography images.

Several types of ablation techniques are known. For example, radio frequency (RF) ablation may be used during intervention to treat tumorous tissue. Typically, a RF ablation needle produces a high frequency alternating current between 100 kHz and 500 kHz. Ions are agitated by the induced electromagnetic field and due to friction this motion is converted into heat. The heat in turn may induce cell death and hence may result in the destruction of tumor cells.

However, heat propagation may be difficult to predict because it may depend strongly on morphology of the heated tissue and whether for instance blood vessels acting as a heat sink are present. Therefore, it may be almost impossible for a surgeon to tell whether a tumor has been completely treated, especially because such ablation progression generally is not visible under normal ultrasound vision.

WO 2008/023321 A2 describes an interventional device for RF ablation for use in a RF electrical and/or magnetic field especially of a MR imaging system comprises an ablation catheter which is preferably trackable or can be guided or visualized in the image generated by the MR imaging system by means of a micro-coil. However, the alternative of using magnetic resonance imaging (MRI) for guidance of the ablation needle could make the intervention very costly and impractical because then during the surgical intervention such surgery equipment needs to be available.

### SUMMARY OF THE INVENTION

There may be a need for an interventional ablation device and a computer program element allowing simple and cost-effective monitoring of an ablation procedure.

Such need may be met by the subject-matter of the independent claims. Advantageous embodiments are described in the dependent claims.

According to a first aspect of the present invention, an interventional ablation device comprises an ablation needle with an elongated body, an ablation element and at least one sensor element. Therein, the device is adapted for detecting physiological information of tissue surrounding an ablation site based on measurement values provided by the sensor.

According to a second aspect of the present invention, an ablation needle with an elongated body, an ablation element and at least one sensor element is proposed. The sensor is adapted for providing measurement values enabling a detecting of physiological information of tissue surrounding an ablation site. Preferably, the needle comprises two or more sensors at opposite sides of the ablation element.

According to a third aspect of the present invention a computer program element is adapted for enabling, when executed on a computer, to control the following processes during an ablation procedure: acquiring measurement values provided by a sensor arranged on an elongated body of an interventional ablation device, and providing physiological information based on the acquired measurement values. Preferably, an ablation element provided on the body may be controlled based on the physiological information or the physiological information may be displayed to a user.

A gist of the present invention may be seen in the idea to integrate one or more sensors into the elongated body of an interventional ablation device which sensor(s) allows to detect physiological information of tissue which is treated using the ablation element for example during a surgical ablation intervention. From the detected physiological information, it may then be possible to discriminate a type of tissue adjacent to the sensor, i.e., whether the tissue surrounding the body of the ablation device adjacent to the sensor is for example normal healthy tissue, tumorous tissue or ablated tissue. Such tissue discrimination information may then be provided to a surgeon or may be used to automatically control an ablation procedure. For example, knowing a volume and geometry of a tumor enclosed within healthy tissue for example by preceding acquisition of computer tomography information and furthermore knowing a precise location of the ablation device with respect to the tumor as well as a precise location of the sensor with respect to the ablation element of the ablation device, an ablation procedure may be precisely controlled allowing to completely destroy the tumor without unnecessarily affecting adjacent healthy tissue.

The sensor may be for example an optical sensor possibly connected to or comprising a light source and a light detector. The sensor may then be adapted to provide measurement values based on light reflected by adjacent tissue. Advantageously, the sensor may be adapted for measuring a reflectance spectrum of the reflected light. From such reflectance spectrum, the type of tissue may be derived. Other types of optical sensors using optical techniques such as fluorescence detection, two-photon spectroscopy, Raman spectroscopy, differential path length spectroscopy or diffuse optical tomography may be used as well for detecting the physiological information of the adjacent tissue. Furthermore, the sensor may be adapted for microscopic sensing like using fiber bundle approach, scanning optical coherence tomography or scanning fiber technology.

Advantageously, at least two sensors are arranged at opposing sides of the ablation element, preferably along a line parallel to the longitudinal axis of the elongated body. Such two sensors may be arranged adjacent to the ablation element and at a predetermined distance apart from the ablation element. Having such two sensors arranged at opposing sides of the ablation element may allow for monitoring an ablation progression in both opposing directions away from the ablation element and parallel to the elongated body.

Advantageously, a plurality of sensors is arranged at opposing sides of the ablation element. The sensors may be arranged along a line parallel to the longitudinal axis of the elongated body and may be spaced apart from each other at predetermined distances. By monitoring the measurement values provided by each of the spaced apart sensors, an ablation progression may be monitored and the ablation process may be stopped as soon as the entire tumorous tissue has been ablated and before an excessive amount of healthy tissue is affected.

In further embodiments, the ablation device may additionally comprise a controller for automatically controlling the ablation element based on the detected physiological information. For example, such controlling may be based on measurement values of specific sensors out of a plurality of sensors indicating that during the ablation procedure, tissue adjacent to the sensor changes optical properties due to a transition from tumorous tissue to ablated tissue while neighboring sensors detect healthy tissue or a transition from healthy tissue to ablated tissue. Such information may then be used to stop the ablation procedure.

Additionally, the controller may take into account additional information on tissue at the ablation site obtained in pre-operative data acquisition such as e.g. information on a geometry or volume of tumorous tissue obtained by e.g. a preceding MRI analysis.

Furthermore, the ablation device may comprise an imaging device for acquiring a plurality of measurement values provided by one or more sensors in different orientations of the respective sensor and generating there from a two-dimensional image. Furthermore, having generated a plurality of two-dimensional images at different locations of the respective sensor, a three-dimensional image may be generated using e.g. tomographical techniques.

It is to be noted that aspects and embodiments of the present invention are described herein with reference to different subject-matters. In particular, some embodiments are described with reference to the interventional ablation device and its components such as particularly an ablation needle whereas other features are described with reference to specifically using or controlling such interventional ablation device. However, a person skilled in the art will gather from the above and the following description that, unless other notified, in addition to any combination of features belonging to one type of subject-matter also any combination between features relating to different subject-matters is considered to be disclosed with this application.

### BRIEF DESCRIPTION OF THE DRAWINGS

Features and advantages of the present invention will be further described with reference to specific embodiments as shown in the accompanying figures but to which the invention shall not be limited.

FIG. 1 schematically shows an interventional ablation device according to an embodiment of the present invention.

FIGS. 2 to 4 schematically show a progression of an ablation procedure using an ablation device according to an embodiment of the present invention.

FIGS. 5 to 7 show examples of reflectance spectra measured by an optical sensor as it may be used in an ablation device according to an embodiment of the present invention.

The features shown in the drawings are schematic only and are not to scale. Throughout the figures, similar features are indicated with similar reference signs.

### DETAILED DESCRIPTION OF EMBODIMENTS

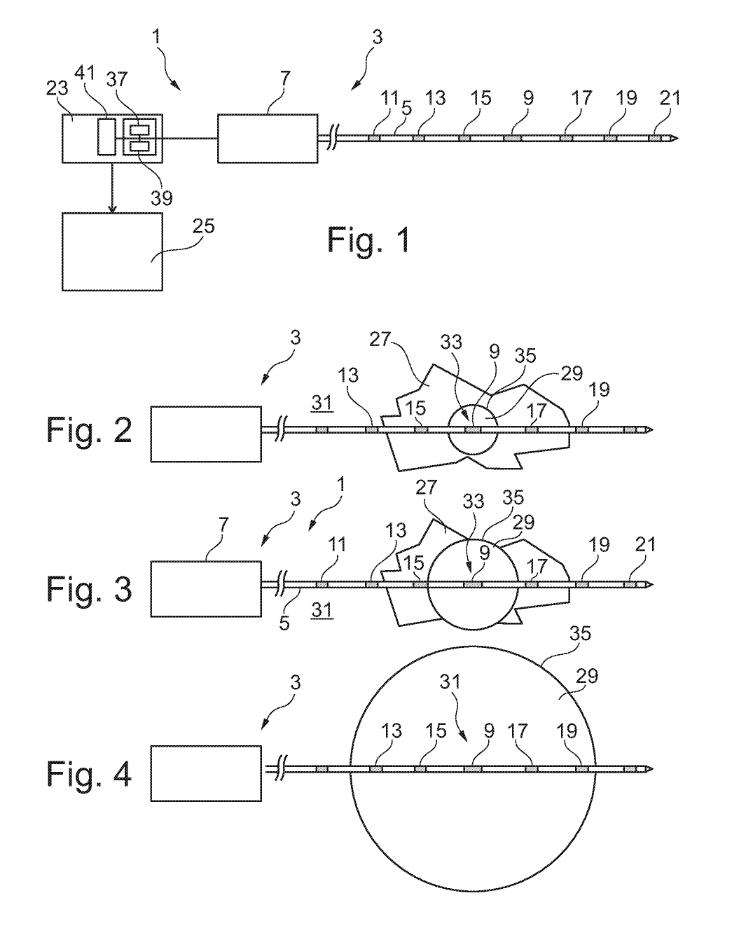

FIG. 1 shows an interventional ablation device 1 according to an embodiment of the present invention. The ablation device 1 may be used for example to ablate, i.e. remove or destroy, e.g. malicious tissue such as tumorous tissue enclosed by healthy tissue.

The ablation device 1 comprises an ablation needle 3 having an elongated body 5 and a handle 7. The elongated body 5 has a small diameter of e.g. between 22 and 11 gauge i.e. 0.72 and 3.05 mm and a length of e.g. between 100 and 300 mm or even more preferred between 120 and 250 mm. Furthermore, the elongated body 5 has a pointed tip at a distal end thereof thereby enabling to introduce the ablation needle easily into a patient's tissue.

An ablation element 9 is arranged on the body 5. The ablation element 5 is arranged in a region close to the distal end of the body 5 but spaced apart from this distal end. For example, the ablation element may be arranged at a distance between 5 and 100 mm away from the distal end of the body 5.

At opposite sides along the body 5, a plurality of sensors 11, 13, 15, 17, 19, 21 are arranged on the body 5. The sensors are spaced apart from each other and from the ablation element 9 at distances of e.g. between 1 and 50 mm or even more preferred between 1 and 10 mm.

The ablation needle 3 may be provided as a disposable product. Such disposable needle may be connected to further components of the ablation device. For example, the needle 3 may be connected to a controller console 23 which may acquire measurement values from the sensors provided on the needle and may control the ablation element provided on the needle. The needle may be disposed and replaced after each operation.

The ablation element 9 may be a radio frequency ablation (RFA) element adapted to emit energy by producing a high frequency alternating current in a range of 100 kHz to 500 kHz. Such high frequency energy may be absorbed by ions comprised in the adjacent tissue and due to an agitation of these ions, the tissue may be effectively heated. The heat may induce cell death. Accordingly, when the ablation needle is inserted into a patient's tissue such that its ablation element 9 is located within tumorous tissue, such tumorous tissue may be locally heated and the tumor cells may be destroyed.

Alternatively, the ablation element may rely on other ablation principles such as e.g. cryoablation. In cryoablation, adjacent tissue is cooled down and tumor cells may be killed by icing.

As schematically shown in the sequence of FIGS. 2 to 4, the ablation needle 3 may be inserted into tumorous tissue 27 enclosed by normal healthy tissue 31 such that the ablation element 9 is located approximately in the center of the tumorous tissue 27. A correct positioning of the ablation needle 3 may be monitored e.g. using external imaging means such as ultrasound imaging or computer tomography. After starting an ablation procedure, the ablation element 9 heats or cools adjacent tissue within an ablation site 31. The ablation volume 35 including ablated tissue 29 around an ablation site 33 in which biological cells have been killed due to excessive heat or icing grows with continuing ablation progress.

Conventionally, a surgeon has not been able to monitor the progress of the ablation, i.e. to monitor whether the lesion generated during the ablation procedure has already destroyed the entire tumor or not and whether healthy tissue is started to be damaged. As the heat/icing propagation is difficult to predict because it typically depends strongly of the morphology of the adjacent tissue and whether for instance blood vessels are present, the surgeon had to rely on his experience and frequently not the entire tumor has been destroyed or, on the other side, excessive healthy tissue has been damaged during an ablation procedure.

In order to overcome such deficiencies, the ablation device 1 proposed herein comprises a multiplicity of sensors 11, 13, 15, 17, 19, 21 arranged along a longitudinal direction on the elongated body 5 on both sides of the ablation element 9. Each of the sensors 11-21 may measure parameter values which may be used to indicate physiological information concerning adjacent tissue 27, 29, 31 such as indicating whether the adjacent tissue is healthy tissue 31, tumorous tissue 27 or ablated tissue 29.

For this purpose, the sensors 11-21 may be provided as optical sensors adapted for measuring a reflectance spectrum of light reflected by the adjacent tissue. The optical sensor may comprise an optical fiber (not shown in the figures for clarity reasons). A distal end of the optical fiber may be arranged at the distal end of the body 5 thus forming a local sensor 11-21. A proximal end of the optical fiber may be connected to a control console 23. In the console 23, a light source 37 such as an LED and a light detector 39 may be provided. Light coming from the light source 37 may be coupled into the optical fiber at the proximal end and may propagate towards the distal end where it exits the optical fiber and may illuminate adjacent tissue. Light that is back-reflected towards this fiber may then be captured by the fiber and guided to the detector 39 at the fiber proximal end. Using such system comprising a light source 37, optical fiber and a light detector 39, the reflectance spectrum may be acquired. Particularly, the reflectance spectrum may be measured in the visible and/or near infrared range.

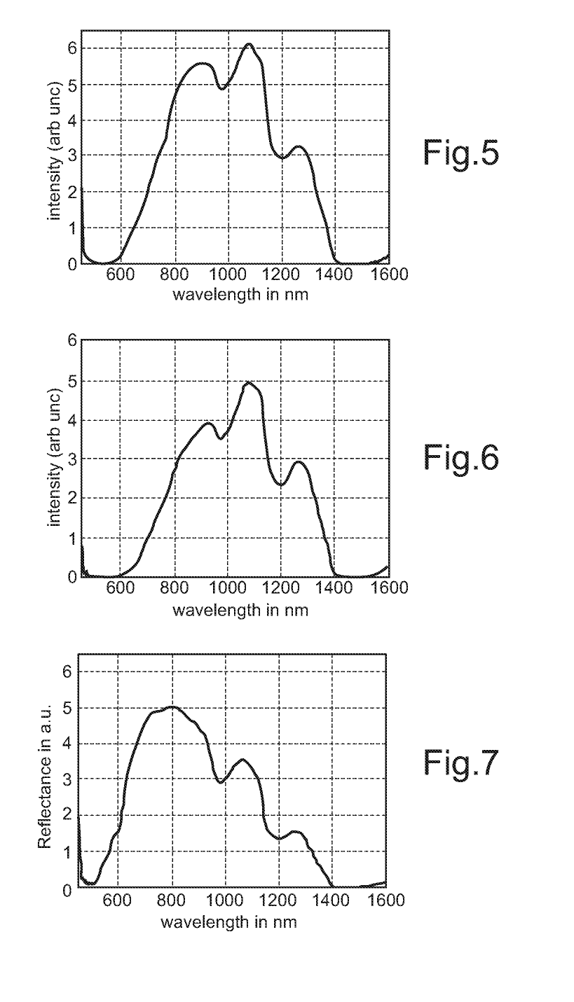

FIGS. 5 to 7 show reflectance spectra as they may be detected by the detector comprised in one of the sensors 11-21. Such spectra as acquired with reflectance spectroscopy may have typical characteristics depending on the type of tissue 27, 29, 31 and in particular of its physiological properties. For example, the reflectance spectrum shown in FIG. 5 indicates normal healthy liver tissue 31. The reflectance spectrum shown in FIG. 6 represents tumorous liver tissue 27. The reflectance spectrum shown in FIG. 7 represents ablated liver tissue 29. Accordingly, from the measurements provided by the sensors 11-21 and the reflectance spectra obtained therewith, physiological information of tissue surrounding an ablation side 33 may be derived.

Coming back to FIGS. 2 to 4, an ablation control scheme may be explained. At the beginning of the ablation process, the RF ablation element 9 is provided with electrical energy which is then transformed to RF energy heating a volume 35 around the ablation site 33 adjacent to the ablation element 9. At that point in time, none of the sensors 11-21 arranged equidistant along the body 5 at both sides of the ablation element 9 detects ablated tissue 29. The sensors 15, 17 closest to the ablation element 9 are lying within the tumor and therefore detect a reflectance spectrum representing tumorous tissue 27. The sensors 11, 13, 19, 21 further away from the ablation element 9 are positioned outside the tumor and therefore detect a reflectance spectrum indicating normal healthy tissue 31.

With progressing ablation process, the volume 35 of the ablation site 33 increases. As shown in FIG. 3, the ablated volume 35 reaches the innermost sensor 15, 17 after a while but does not yet reach the outer sensors 13, 19. This indicates that the ablation is not sufficient yet. In FIG. 4, the ablated volume 35 has also reached the sensors 13, 19. These sensors 13, 19 have originally detected a reflectance spectrum indicating healthy tissue 31 and now measure a reflectance spectrum indicating ablated tissue 29. Based on such monitoring result, the ablation device may detect that the ablated volume 29 covers the entire tumor and that the ablation process may be stopped. Such information may either be indicated to a surgeon via a display 25 connected to the console 23 of the interventional ablation device 1 or may be used internally in the console 23 in order to automatically stop an energy supply to the ablation element 9 thereby stopping the ablation process.

In the above described embodiment, physiological information is acquired along a one-dimensional cross-section of the tumor using the sensors 11-21 arranged along the body 5 of the ablation device 1. Since the tumor may be irregular, i.e. larger in a direction perpendicular to the longitudinal direction of the elongated body 5, it may be advantageous to combine the information provided by the ablation device 1 with pre-operative data acquired prior to the insertion of the ablation device by using other imaging modalities such as e.g. CT or MRI. From such pre-operative data, the dimension of the tumor in various directions may be deduced. The information that the tumor is smaller in a direction parallel to the longitudinal direction of the inserted elongated body 5 than in a direction perpendicular thereto may be used to correct or adapt the controlling of the ablation procedure. As the dimension of the elongated body 5 and the position of the sensors 11-21 relative to the ablation element 9 are known, the size of the tumor along the elongated body 5 may be measured. Using the size perpendicular to the body's 5 longitudinal direction coming from the pre-operative data, the ablation process may be suitably controlled and stopped as soon as the ablated volume 29 has reached a size that is larger than the largest dimension of the tumor.

According to a further embodiment, the ablation device 1 further comprises an imaging device 41 possibly comprised in the console 23. During an ablation process, the ablation needle 3 may be rotated about a longitudinal axis of the body 5. Thereby, although the sensors 11-21 are positioned only along one dimension and each of the sensors is adapted to acquire a measurement value at one adjacent point in space only, a two-dimensional imaging may be enabled by acquiring a plurality of measurement values using the sensors when arranged in different orientations. Accordingly, a 2D image containing physiological information on adjacent tissue may be acquired from measurement values of the rotated sensors 11-21. Using tomographic algorithms such as e.g. those that have been developed for diffuse optical tomography (DOT), even three-dimensional imaging may be possible by acquiring a plurality of 2D images generated at different locations of the sensors 11-21.

Finally, it is to be noted that instead of or additional to measuring reflectance spectra, the sensors 11-21 may be adapted for measuring other parameters indicative of physiological information using for example fluorescence detection, two-photon spectroscopy. Raman spectroscopy, differential path length spectroscopy or diffuse optical tomography. Furthermore, sensors may be capable of microscopic sensing like using fiber bundle approach, scanning optical coherence tomography or scanning fiber technology. Furthermore, apart from optical sensors, also other sensors like temperature sensors, PH sensors, stiffness sensors or ultrasound transducers may be arranged along the elongated body 5 of the ablation device 1 in order to complement the optical sensors 11-21. The ultrasound technique can be combined with optical methods like photoacoustic detection.

It should be noted that the term “comprising” and similar does not exclude other elements or steps and that the indefinite article “a” does not exclude a plurality of items. Also elements described in association with different embodiments may be combined. It should be furthermore noted that reference signs in the claims shall not be construed as limiting the scope of the claims.

### LIST OF REFERENCE SIGNS

1 Ablation device
3 Ablation needle
5 Elongated body
7 Handle
9 Ablation element
11 Sensor
13 Sensor
15 Sensor
17 Sensor
19 Sensor
21 Sensor
23 Console
25 Display
27 Tumorous tissue
29 Ablated tissue
31 Normal tissue
33 Ablation volume
35 Ablation site
37 light source
39 light detector
41 imaging device

1-15. (canceled)

16. An interventional ablation system, comprising:
an ultrasound imaging device and
an ablation needle, the ablation needle comprising:
an elongated body;
an ablation element; and
at least one optical sensor configured to detect physiological information of tissue surrounding an ablation site and
at least one ultrasound transducer arranged along the elongated body;
wherein the at least one sensor configured to detect physiological information measures a reflectance spectrum of light reflected by the tissue; and
wherein the system is configured to discriminate from the reflectance spectrum a type of tissue adjacent to the sensor and display an image of the tissue.

17. The system of claim 16 configured to acquire a plurality of measurement values provided by the optical sensor in different orientations of the sensor and to generate a 2D image from the acquired measurement values.

18. The system of claim 17 adapted to generate a 3D image from a plurality of 2D images generated at different locations of the sensor.

19. The system of claim 18 wherein the ablation needle comprises a sensor adapted to measure parameters of diffuse optical tomography.

20. The system of claim 16 wherein the system is adapted for at least one of fluorescence detection, two-photon spectroscopy, Raman spectroscopy, differential path length spectroscopy, diffuse optical tomography and microscopic sensing.

21. An interventional ablation system, comprising:
an imaging device and
an ablation needle comprising:
an elongated body;
an ablation element; and
at least one optical sensor configured to detect physiological information of tissue surrounding an ablation site;
wherein the system is configured to discriminate a type of tissue adjacent to the sensor and display an image of the tissue; and
wherein the system is configured to acquire a plurality of measurement values provided by the optical sensor in different orientations of the sensor and to generate a 2D image from the acquired measurement values.

22. The system of claim 21 adapted to generate a 3D image from a plurality of 2D images generated at different orientations of the sensor as the interventional ablation device is rotated about an axis of the elongated body.

23. The system of claim 21, wherein the system is adapted for at least one of fluorescence detection, two-photon spectroscopy, Raman spectroscopy, differential path length spectroscopy, diffuse optical tomography and microscopic sensing.

24. A method of monitoring progress of an interventional ablation comprising:
acquiring measurement values of physiological information from a sensor arranged on a longitudinal axis of an elongated body of an interventional ablation device during an ablation procedure;
providing physiological information of tissue surrounding an ablation site based on the acquired measurement values; and
discriminating a type of tissue adjacent to the sensor.

25. The method of claim 24 further comprising a step of acquiring data from an ultrasound transducer arranged on the elongated body of the interventional ablation device.

26. The method of claim 24 further comprising a step of acquiring a plurality of measurement values provided by the sensor in different orientations of the sensor as the interventional ablation device is rotated about the axis to generate a 2D image from the acquired measurement values.

27. The method of claim 26 further comprising a step of generating a 3D image from a plurality of 2D images generated at different locations of the sensor.

28. The method of claim 27 wherein the step of generating a 3D image from the plurality of 2D images generated at different locations of the sensor comprises using an algorithm developed for diffuse optical tomography.
